(19) United States
(12) Patent Application Publication
(10) Pub. No.: US 2003/0225460 A1
(43) Pub. Date: Dec. 4, 2003

(54) Compositions for generating submucosal fluid cushions

(76) Inventors: Christopher J. Gostout, Rochester, MN (US); Arnaldo B. Feitoza, Telemaco Borba-Pr (BR)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/376,815

(22) Filed: Feb. 28, 2003

Related U.S. Application Data
(60) Provisional application No. 60/360,700, filed on Feb. 28, 2002.

(51) Int. Cl. A61F 2/02

(52) U.S. Cl. 623/23.72

(57) ABSTRACT

Methods for creating submucosal fluid cushions using methylcellulose are provided, as well as compositions and articles of manufacture for generating submucosal fluid cushions.

### CROSS-REFERENCE TO RELATED APPLICATIONS

This application claims priority from U.S. Provisional Application Serial No. 60/360,700, filed Feb. 28, 2002.

### TECHNICAL FIELD

This invention relates to endoscopic surgery, and more particularly to facilitating endoscopic mucosal resection and/or gastrointestinal polypectomy with a submucosal fluid cushion.

### BACKGROUND

Injection of fluid into the submucosa frequently is used to facilitate surgical procedures such as endoscopic mucosal resection (EMR) and polypectomy in the gastrointestinal tract (Ishiguro et al. (1999) Gastrointest. Endosc. 50(3):329-333). A submucosal fluid cushion (SFC) can be used to lift the section of mucosa to be resected, isolate the lesion, and protect the muscularis propria from injury. The piecemeal removal of large sessile polyps without injection can result in deep thermal ulceration involving the muscularis propria (Iishi et al. (1997) Hepatogastroent. 44:698-702). The piecemeal resection of early esophageal cancers also can result in bleeding and perforation (Kodama and Kakegawa (1998) Surgery 123:432-439).

SFC created with fluids such as normal saline and glucose 50% can be short-lived, and require an expedient approach to the targeted area. In addition, repeated injections frequently are necessary during piecemeal resections in an attempt to keep the lesion isolated from the submucosa and to prevent delayed injury to the underlying tissue (Sakai et al. (1996) Gastrointest. Endosc. 44(1):65-68). Stenosis can be another complication of using such solutions (Conio et al. (2001) Endosc. 9:791-794). Other fluids such as sodium hyaluronate may result in a longer lasting SFC (Yamamoto et al. (1999) Gastrointest. Endosc. 50(2):251-256), but these fluids typically are costly and may not be readily available. A long-lasting SFC would benefit EMR and piecemeal resection of larger sessile polyps, particularly in cases involving multiple endoscopic accessory exchanges or lengthy procedures.

### SUMMARY

This invention is based on the discovery that compositions containing methylcellulose (e.g., hydroxypropyl methylcellulose (HPMC) or carboxymethylcellulose) can be used to create a relatively long-lasting submucosal fluid cushion in a subject. Methylcelluloses such as HPMC have suitable viscoelastic characteristics, and generic HPMC is relatively inexpensive and readily available.

In one aspect, the invention features a method of generating a submucosal fluid cushion in a subject. The method can include injecting an amount of a methylcellulose solution into the submucosa of the subject, wherein the amount is effective to generate a submucosal fluid cushion. The methylcellulose can be HPMC or carboxymethylcellulose. The submucosa can be in the gastrointestinal tract. The subject can be a mammal (e.g., a human). The concentration of methylcellulose can be from about 0.1% to 1.5% (e.g., from about 0.3% to 1.0%, or 0.83%). The amount can be from about 1 to 15 ml (e.g., from about 3 to 12 ml, or from about 4 to 6 ml). The method can further include performing endoscopic mucosal resection or gastrointestinal polypectomy. The method can further include injecting a dye (e.g., methylene blue or indigo carmine) and/or a local vasoconstrictor (e.g., epinephrine) into the submucosa of the subject. The methylcellulose and the dye or the local vasoconstrictor can be injected simultaneously into the submucosa of the subject.

In another aspect, the invention features compositions containing methylcellulose and a local vasoconstrictor, methylcellulose and a dye, or methylcellulose, a local vasoconstrictor, and a dye. The methylcellulose can be HPMC.

In another aspect, the invention features an article of manufacture containing: (a) a syringe containing a methylcellulose solution, and (b) instructions for using the methylcellulose solution to generate a submucosal fluid cushion in a subject. The methylcellulose can be HPMC. The syringe can further contain a dye and/or a local vasoconstrictor. The syringe can contain between about 2 and about 20 ml of methylcellulose solution. The article of manufacture can further contain a plurality of syringes, wherein each syringe in the plurality contains methylcellulose solution.

In still another aspect, the invention features a method of performing endoscopic mucosal resection or colonic polypectomy on a subject having a mucosal abnormality. The method can include: (a) generating a submucosal fluid cushion in the subject by injecting a methylcellulose solution into the submucosa of the subject, and (b) resecting the mucosal abnormality. The methylcellulose can be HPMC.

The invention also features the use of methylcellulose for the manufacture of a medicament for generating an SFC in a subject. Methylcellulose such as HPMC can be placed into a device such as a syringe. In addition, methylcellulose can be combined with one or more other components (e.g., a dye and/or a local vasoconstrictor).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DETAILED DESCRIPTION

Perforation and hemorrhage are potential complications of endoscopic ablation and removal of sessile lesions. Prophylactic methods for injecting fluid into the submucosa to create a fluid cushion between a lesion and the deeper layers of the gut wall are useful for preventing such complications. As such, formation of SFC has become an integral part of endoscopic mucosal and polyp resection. As used herein, an SFC is a pocket of fluid that isolates the mucosa from the underlying tissue. An SFC is useful, for example, to isolate a mucosal abnormality that is to be removed (e.g., a colonic polyp, an esophageal lesion, or other dysplasia) from underlying tissue, such that the underlying tissue is protected from damage during resection of the abnormality. This procedure can be particularly useful during the resection of flat lesions that otherwise may be difficult to access.

Fluids typically used for injection into the submucosa of the gastrointestinal tract can be costly and often create only short-lasting SFC. As described herein, fluids (e.g., solutions or suspensions) containing a methylcellulose can be used to create long-lasting SFC with minimal tissue reaction, and provide a low-cost alternative to other fluids used to create SFC. In addition, methylcelluloses can be combined with other components such as dyes or local vasoconstrictors in order to facilitate visualization of SFC and resection of an abnormality. A single SFC can be used during resection of a single abnormality or multiple abnormalities that are closely spaced. A single SFC also can be useful for resecting a large lesion that must be removed in a piecemeal manner (i.e., removed in more than one fragment). Alternatively, multiple SFC can be generated in the same patient to facilitate the resection of multiple abnormalities.

Methods for Generating SFC

The invention provides methods for using solutions containing methylcelluloses to generate an SFC in a subject (e.g., a mammal such as a human) undergoing EMR to remove abnormalities such as a colonic polyp or an esophageal lesion, for example. In some embodiments, a suspension of methylcellulose particles can be used to create an SFC in a subject.

Suitable types of methylcellulose are pharmaceutically acceptable for use in mammalian subjects (e.g., humans). In addition, solutions of suitable types of methylcellulose have viscoelastic properties appropriate for generation of long-lasting SFC. Methylcelluloses useful for creating long-lasting SFC can be identified by, for example, testing in an animal such as a pig, as described in the Examples below. Non-limiting examples of suitable methylcelluloses include HPMC and carboxymethylcellulose. As described in Example 2, HPMC is particularly useful for creating long-lasting SFC.

Suitable concentrations of methylcellulose can range from about 0.01% to about 5% (e.g., 0.05%, 0.1%, 0.25%, 0.5%, 1%, 2%, 2.5%, 3%, 4%, or 4.5%). A methylcellulose such as HPMC, for example, typically is used at concentrations that range from about 0.1% to about 1.5%. Non-limiting examples of useful HPMC concentrations include 0.2%, 0.3%, 0.4%, 0.5%, 0.6%, 0.7%, 0.83%, 1.0%, 1.25%, and 1.5%. HPMC can be obtained commercially from vendors such as Akorn, Inc. (Decatur, Ill.), for example.

An SFC can be generated by injecting a suitable amount of fluid underneath the mucosa (e.g., under the inner lining of the intestinal tract). Such injections can be accomplished with standard syringes and needles (e.g., 1, 3, 6, or 12 cc syringes and 21-gauge, 23-gauge, or 25gauge needles). Injection of more viscous fluids may be facilitated by the use of larger gauge needles (i.e., needles having a lower numbered gauge designation). Catheter needles also may be used.

Suitable amounts of fluid elevate the selected region of the mucosa (typically containing an abnormality such as a polyp or lesion) from the underlying tissue, to an extent deemed sufficient by a clinician conducting or assisting with the procedure (e.g., resection). A fluid injection that raises the mucosa into the lumen to a height that is about 30 to about 35 percent of the lumen diameter can be particularly useful. The amount of fluid injected typically is dependent on the size of the lesion, with larger lesions requiring larger amounts of fluid. Non-limiting examples of useful fluid amounts include 1 ml, 2 ml, 3 ml, 4 ml, 6 ml, 9 ml, 12 ml, 15 ml, and 20 ml.

In some embodiments, SFC can be generated using solutions that contain components in addition to methylcellulose. These include, without limitation, dyes such as methylene blue or indigo carmine, and local vasoconstrictors such as epinephrine. The inclusion of a dye can be used to facilitate observation of the size and integrity of an SFC, while a local vasoconstrictor can be used to prevent or reduce excessive bleeding at the site of resection. A dye and/or a vasoconstrictor can be mixed with a methylcellulose solution before generation of an SFC. Alternatively, a methylcellulose solution can be injected separately from a dye or a vasoconstrictor. One of ordinary skill in the art (e.g., a clinician or medical technician) can readily determine suitable concentrations of dyes and vasoconstrictors to include in solutions or suspensions used to create SFC. A methylcellulose solution can contain, for example, about 0.1 percent to about 5 percent dye (e.g., 0.1, 1.0, 1.5, 2.3, 2.5, 3, 4, or 4.5 percent dye), and/or about 0.1 to about 10 percent vasoconstrictor (e.g., 0.1, 1, 2, 3, 3.5, 4, 5, 5.75, 6, 7, 8, 9, or 9.5 percent vasoconstrictor).

Compositions

The invention provides compositions that can be used to generate an SFC in a subject undergoing an EMR, for example. Such compositions can include a methylcellulose (e.g., HPMC) as well as components such as dyes and/or local vasoconstrictors. The methylcellulose and other components can be present in amounts and concentrations as described above, which are suitable for creating an SFC in a subject. In one embodiment, a composition can be dried (e.g., lyophilized). Such a composition can be prepared for injection by dissolving or suspending the dried composition in a diluent or pharmaceutically acceptable carrier that is appropriate for administration to a subject. In another embodiment, a composition can be in liquid form (e.g., in a pharmaceutically acceptable carrier). As used herein, a “pharmaceutically acceptable carrier” is a pharmaceutically acceptable solvent, suspending agent, or any other pharmacologically inert vehicle for delivering one or more compounds (e.g., a methylcellulose with or without a dye and/or a vasoconstrictor) to a subject undergoing EMR. Pharmaceutically acceptable carriers can be selected with the planned manner of administration in mind so as to provide for the desired bulk, consistency, and other pertinent transport and chemical properties, when combined with one or more components of a given pharmaceutical composition. Compositions and formulations for administration by submucosal injection can include sterile aqueous solutions, which also can contain buffers, diluents and other suitable additives (e.g., carrier compounds and other pharmaceutically acceptable carriers). Typical pharmaceutically acceptable carriers for use with injectable compositions include, by way of example and not limitation, water and saline solution.

Compositions described herein can be combined with packaging material and sold as articles of manufacture or kits. Components and methods for producing articles of manufacture are well known. In some embodiments, an article of manufacture may contain a composition in combination with other components (e.g., syringes) that can be used to generate an SFC in a subject. Such articles of manufacture may include a single syringe or a plurality of syringes containing a composition (e.g., a solution of HPMC) that is useful for generating an SFC, as well as needles and any other desirable components. In addition to HPMC, the composition also can contain other compounds (e.g., a dye or a local vasoconstrictor). The syringes and needles can be of any appropriate size as defined above (e.g., 6 cc syringes and 23-gauge needles), and the amount of fluid contained therein can be any suitable amount (e.g., about 1 ml, 2 ml, 3 ml, 4 ml, 6 ml, 9 ml, 12 ml, 15 ml, or about 20 ml). Each of the components can be sterilized and ready to use. A label or instructions describing how the compositions or fluid-filled syringes can be used to generate an SFC in order to facilitate a medical procedure such as EMR or colonic polypectomy can be included in such articles of manufacture or kits.

The invention will be further described in the following examples, which do not limit the scope of the invention described in the claims.

### EXAMPLES

### Example 1

### Materials and Methods

HPMC study: This study was approved by the Mayo Foundation Institutional Animal Care and Use Committee. A total of 12 white-domestic pigs (60 kg) divided into 2 groups underwent the creation of 36 SFC in the esophagus. The esophagus was chosen as the test site due to ease of animal preparation, technical ease, and the potential for unobstructed, prolonged endoscopic viewing.

Endoscopy was performed in fasting pigs placed in the left lateral decubitus position after tracheal intubation and general anesthesia. The procedures were performed with an esophagogastroduodenoscope (model GIF-100 or GIF-XQ10; Olympus America Inc., Melville, N.Y.). Three distinct SFC were sequentially created in the middle and distal esophagus of each animal by injecting 0.83% HPMC (Akorn, Inc.) into the submucosa through a 23-gauge needle (Hobbs Medical, Inc., Stafford Springs, Conn. and Olympus America Inc.) attached to a balloon inflation syringe (Boston Scientific, Watertown, Mass.). To create the SFC, a single tangential injection in the submucosa of the target area was attempted. After puncturing the tissue, HPMC was slowly injected to create a large, visible semitranslucent fluid cushion that projected above the mucosa to a height estimated to be one third the diameter of the lumen. If the mucosa did not elevate after injection of 2 to 3 ml of HPMC, the needle was repositioned within the tissue while maintaining a continuous, slow injection of HPMC. If the needle fell out, a new puncture was made close to the first. After creating a visually adequate SFC, the catheter was kept in position for a short time to block the puncture site and prevent premature escape of HPMC into the lumen. The injection was halted once an adequate SFC was created. The volume recorded for creation of each SFC was the total volume of HPMC used, including HPMC misplaced deep in the muscularis propria or lost in the lumen.

An assisting nurse recorded the duration of each SFC, beginning immediately after each injection at the time the needle was withdrawn from each SFC. The endoscope was left in the esophagus to observe and time the duration of each SFC for up to 45 minutes. SFC were considered present if any semitranslucent mucosal elevation remained evident. Conversely, SFC were considered resolved when the mucosal elevation became flattened and opaque. SFC sites were individually observed and timed continuously after each injection. For each SFC, the volume injected, bleb length, distance from the esophagogastric (EG) junction, and distance from the incisors were noted. The intention was to underestimate rather than overestimate the duration of the blebs.

The SFC sites in the first group of animals (group I) were tattooed with 0.5 ml of carbon particle based dye (Spot, GI Supply, Camp Hill, Pa.) placed within approximately 1-2 cm of the cephaled extent of the SFC at the end of the procedure. SFC sites in the second group (group II) were marked by an endoscopically placed suture (Endocinch, C R Bard, Murray Hill, N.J.) located 4 cm proximal to the upper margin of each SFC. The distance from this stitch to the EG junction was measured. All procedures were video recorded and endophotos were taken.

Animals were allowed to recover from anesthesia and feed ad libitum. After seven days, the animals underwent esophagogastroduodenoscopy (EGD) to assess the SFC sites. Each procedure was video recorded and endophotos were taken. Animals were immediately sacrificed and necropsied to assess the esophagus and mediastinum. The esophagus and proximal stomach were resected and then fixed in 10% buffered formalin for histopathology. A longitudinal incision was made to open the esophagus and stomach and allow mounting. This permitted identification of SFC sites using measurements from the EG junction, bleb length, proximal sutures, and tattoos. Statistical comparisons were made between the groups using a two-tailed Fisher exact test for categorical data. A p value <0.05 was considered significant.

SFC generation using five different fluids: This study was approved by the Mayo Foundation Institutional Animal Care and Use Committee. Groups of five 60-kg female pigs were used for each of the five test solutions. In each pig, six 5 ml submucosal injections of a single test solution were performed at separate sites within the distal half of the esophagus.

Each fasting animal underwent an EGD as described above, under general anesthesia and with endotracheal intubation. Catheter injection needles (23-gauge; Hobbs Medical, Inc.) were used to perform the injections.

Five solutions were studied: normal saline (NS), normal saline containing 1:10,000 epinephrine (NSE), 50% dextrose (D50), rooster comb derived hyaluronic acid (HA; Sigma, St. Louis, Mo.), and generic “Glyceol” (G). Glyceol, commercially available in Japan (formulation assistance provided by Chugai Pharmaceutical Co., Tokyo), is a hypertonic solution consisting of 10% glycerol and 5% fructose in normal saline. All solutions were delivered at room temperature. Indigo carmine was added to each solution (at approximately 10% v/v) to better visualize submucosal diffusion of the solution. A submucosal injection was defined as the prompt appearance of a spreading and enlarging bleb of solution with a semi-translucent bluish coloration.

The time for each bleb to disappear was recorded using a stopwatch. Timing began immediately after all 5 ml of the test solution was injected into the submucosa. Timing ended when the bleb flattened completely. Injections were performed sequentially into separate sites, after the complete disappearance and timing of the previous bleb. The investigators were not blinded as to the different solutions, and timing was performed by a Developmental Endoscopy Unit nurse who assisted with the procedures.

The disappearance time for each solution was calculated in terms of median, mean, and standard deviation (SD). In order to test differences among the solutions, data were approximately normally distributed after log transformation, and were tested by means of GLM repeated measures analysis of variance. This procedure tested the effects of the within-subject factors (pigs) and the between-subjects factors (solutions). The post hoc pairwise Tukey's test was performed to compare the solutions. Statistical analysis was performed using the SPSS software package. A two-tailed p value <0.05 was considered statistically significant.

### Example 2

### HPMC is Useful for SFC

HPMC injection through a 23-gauge needle with a balloon dilator syringe was easily performed, and HPMC free in the lumen was readily suctioned and rinsed from the endoscope lens. The injection of HPMC into the submucosal layer induced prompt semitranslucent elevation of the mucosa that remained visible for a mean of 36 minutes (range 3-45 minutes, median 40 minutes) in the group I animals, and 38 minutes (range 5-45 minutes, median 45 minutes) minutes in the group II animals. The mean volume of HPMC injected for group I was 5.22 ml (range 3-10 ml, median 5 ml), while the length of the SFC was 3.55 cm (range 2-5 cm, median 3.5 cm; see Table 1). For group II, these values were 4.5 ml (range 2-11 ml, median 4.5 ml) and 2.83 cm (range 2-4 cm, median 3 cm), respectively. Instances of very short SFC duration typically were due to leakage of HPMC through the needle puncture site, which always was associated with collapse of the elevation. It was observed that slow injection, prolonged occlusion of the puncture site with the needle catheter until the injected volume was accommodated within the submucosa, and a single puncture were associated with increased duration of the SFC.

After one week, all 36 SFC sites had a normal endoscopic appearance. A stained ulcerated nodule was observed in 1 (6%) of the 18 group I (tattooed) SFC sites. Necropsy of the group I animals revealed nodules in 8 (44%) of 18 tattoo sites, including one related to an ulcerated lesion seen at endoscopy. One nodule measured 25 mm and was adhered to the parietal pleura and lung. Tattoo dye also was observed in the lymph nodes. Nodules or other abnormalities were not observed in any of the group II animals by endoscopy or necropsy.

Histological examination of the SFC sites revealed preserved mucosa in all cases, with no residual elevation. Two (6%) of the 36 SFC sites, both from group I, had normal histology. A minimal amount of residual HPMC was suggested in four other SFC sites from group I; three of these sites had nearby tattoo related abnormalities. Residual HPMC was not observed in any of the group II animals. The presence of foamy macrophages was the only histologic finding related to HPMC injection in 27 (75%) of the 36 SFC sites. Foamy macrophages were associated with scattered eosinophils in 3 (8%) of the 36 sites. These were found predominantly in the submucosal layer and occasionally in the muscularis propria and adventia. Focal fibrosis in the adventia was detected in 1 (3%) of the 36 SFC sites, and a subacute abscess was present in another of the 36 sites (in a group I animal).

Two (6%) of the 36 SFC sites, both in the same group II animal, displayed a focal area of fibrinoid necrosis associated with vasculitis. This animal had a difficult intubation and exhibited respiratory distress shortly after anesthesia for the initial procedure; the distress eventually was related to a plugged orotracheal tube. The animal developed hyperthermia and coughing during the post-procedure day, which was diagnosed as a pulmonary infection and improved clinically with the administration of antibiotics. In both cases, the SFC sites looked normal at EGD and necropsy.

In 5 of the 6 group I animals, a mild to moderate lymphohistiocytic response surrounding the dye was present at 8 of the 18 SFC sites, with an abscess associated at 3 sites. Inflammatory reactions related to the tattoo dye ranged from involving all esophageal layers to also involving periesophageal tissue, depending on the tattoo dye location. None of these animals displayed clinical symptoms during survival.

The overall comparison between the groups revealed an unfavorable inflammatory reaction (e.g., presence of acute or subacute abscess, focal vasculitis, or lymphohistiocytic reaction) in 9 (50%) of 18 and 2 (11%) of 18 cushion sites, respectively, in Groups I and II (p=0.027). In 8 of 9 sites in Group I animals, the inflammatory reaction chiefly surrounded the tattoo dye, whereas in Group II animals, no inflammatory reactions were observed that were unrelated to HPMC (8 of 18 vs. 0 of 18; p=0.003). Excluding the inflammatory findings associated with the tattoo dye, there was no statistical difference between Groups 1 and 11 (6% vs. 11%) with regard to the number of unfavorable histologic changes.

TABLE 1
Findings at endoscopy, necropsy, and histopathology after 7 days.

| | Group I | Group II* |
|---|---|---|
| Volume injected (ml) | 5.22 (3-10) | 4.5 (2-11) |
| SFC length (cm) | 3.55 (2-5) | 2.83 (2-4) |
| SFC duration (min) | 36 (3-45) | 38 (5-45) |
| Endoscopy - abnormalities at SFC sites | 0 | 0 |
| Abnormalities unrelated to HPMC | 1/18 (6%)4^{£} | — |
| Necropsy - abnormalities at SFC sites | 0 | 0 |
| Abnormalities unrelated to HPMC | 8/18 (44%)^{££} | — |
| Histopathology - findings at SFC sites | | |
| Normal | 2/18 (11%) | 0 |
| Foamy macrophages (F.M.) only | 15/18 (83%) | 12/18 (67%) |
| F.M. + eosinophils | 0 | 3/18 (17%) |
| F.M + focal fibrosis** | 0 | 1/18 (6%) |
| F.M + subacute abscess | 1/18 (6%) | 0 |
| F.M. + focal vasculitis*** | 0 | 2/18 (11%) |
| Residual HPMC**** | 4/18 (22%) | 0 |
| Histopathology - abnormalities unrelated | | — |
| to HPMC | | |
| Abscess | 3/18 (17%) | — |
| Lymphohistiocytic reaction/fibrosis | 5/18 (28%) | — |
| Total unfavorable histologic reactions | 9/18 (50%) | 2/18 (11%) |

*Stain was not used to mark SFC in group II animals.
**Mild and focal collagen deposition.
***Vessels in the adventia, both in the same animal.
****In all cases, a minimal amount of residual HPMC was suggestive. In three cases, ink related histologic abnormalities were associated.
^{£}Nodule and ulceration surrounding tattoo.
^{££}Nodules, ulceration, mediastinal adhesions or wall thickness

### Example 3

### Duration of SFC Generated with Five Different Test Solutions

The disappearance time (in minutes) for each solution in each pig is shown in Table 2, while mean and median disappearance times for each solution are presented in Table 3. Normal saline resulted in the shortest disappearance time, with a median time of 2.4 minutes and a range of 1.4-4.2 minutes, followed by NSE (median 3.0 minutes, range 1.3-7.3 minutes). The median disappearance times for the other solutions were: D50, 4.7 (2.1-11.6) minutes; G, 4.2 (2.3-13.1) minutes; and HA, 22.1 (9.0-58.2) minutes. The analysis of variance did not show significant within-subject effects (p=0.09). A significant difference between the solutions was observed by testing for between-subjects effects (p<0.0001).

By means of Tukey's test, the disappearance time for NS was significantly shorter when compared to the other solutions (p<0.003), except for NSE. A similar result also was observed for NSE when compared to D50, G, and HA (p<0.01). No difference was detected between D50 and G; both solutions dissipated about two minutes later than NS. The mean disappearance time for HA was approximately 20 minutes longer than NS and NSE and 18 minutes longer than the time for D50 and G, a highly significant difference as compared to all other fluids tested (p<0.0001). The mean disappearance times in the HPMC study, however, were approximately 60% longer than the HA disappearance time, indicating that HPMC is particularly useful for generating SFC.

TABLE 2
Disappearance time of the different solutions in each pig

| Solution | Pig 1 | Pig 2 | Pig 3 | Pig 4 | Pig 5 |
|---|---|---|---|---|---|
| NS | | | | | |
| Mean (SD) | 2.5 (0.5) | 2.8 (0.8) | 2.3 (0.5) | 2.8 (0.7) | 2.4 (0.3) |
| Median (Range) | 2.4 (2.1-3.4) | 2.6 (2.0-4.2) | 2.3 (1.4-3.1) | 2.8 (2.0-4.0) | 2.3 (2.1-3.0) |
| NSE | | | | | |
| Mean (SD) | 3.1 (0.6) | 1.9 (0.5) | 3.9 (2.0) | 2.5 (0.5) | 3.3 (0.6) |
| Median (Range) | 3.1 (2.3-4.1) | 1.8 (1.4-2.4) | 3.9 (1.3-7.3) | 2.5 (1.6-3.3) | 3.5 (2.2-4.1) |
| D50 | | | | | |
| Mean (SD) | 5.1 (1.2) | 5.7 (1.8) | 4.7 (0.7) | 4.7 (3.5) | 6.5 (3.9) |
| Median (Range) | 5.2 (3.4-7.0) | 5.3 (3.0-8.3) | 4.4 (4.1-6.0) | 4.0 (2.1-11.6) | 6.2 (2.6-11.5) |
| G | | | | | |
| Mean (SD) | 3.9 (0.5) | 4.3(0.7) | 3.1 (0.1) | 6.2(3.1) | 8.3(2.7) |
| Median (Range) | 3.9 (3.3-4.5) | 4.1 (3.3-5.2) | 3.1 (3.1-3.3) | 6.8 (2.3-9.2) | 7.7 (5.2-13.2) |
| HA | | | | | |
| Mean (SD) | 22.3 (5.6) | 27.2 (9.2) | 26.6 (18.5) | 17.1 (5.4) | 21.5 (10.4) |
| Median (Range) | 22.4 (15.2-30.1) | 27.6 (12.6-37.1) | 22.2 (13.3-58.2) | 17.5 (9.3-24.0) | 21.9 (9.0-33.1) |

TABLE 3
Disappearance time (minutes) for each solution

| Solution | Mean (SD) | Median | Range |
|---|---|---|---|
| NS | 2.6 (0.6) | 2.4 | 1.4-4.2 |
| NSE | 2.9 (1.2) | 3.0 | 1.3-7.3 |
| D50 | 5.3 (2.5) | 4.7 | 2.1-11.6 |
| G | 5.2 (2.6) | 4.2 | 2.3-13.2 |
| HA | 23.0 (10.5) | 22.1 | 9.0-58.2 |

### Example 4

### SFC Generation with and without Methylene Blue Plus Epinephrine

The use of HPMC to generate SFC was evaluated with methylene blue (MB) and epinephrine (EPI), which can be used to enhance visibility of the target lesion and reduce bleeding during EMR. Nineteen pigs underwent EMR at two different levels in the esophagus. Each animal had one “PURE HPMC” SFC created with 0.83% HPMC alone and a second “HPMC PLUS” SFC created with a solution containing 8 ml of 0.83% HPMC, 0.5 ml of 1:1000 EPI (Abbott Laboratories, North Chicago, Ill.), and 0.2 ml of MB (Faulding Pharmaceuticals, Faulding Puerto Rico, Inc., Aquadilla, PR). Cap-style EMR was performed and the specimen was retrieved from each of the 38 sites. Bleeding was monitored, with significant bleeding defined as prolonged blood loss (>1 minute) or blood loss sufficient to obscure the EMR site and/or abort the procedure. After seven days, each animal underwent EGD and was sacrificed for necropsy and histopathology of the EMR sites.

EMR was successful in each group of SFC, with very similar results. There was no bleeding during or immediately following EMR in either group. SFC volumes were 7.7 (2.3) ml vs. 7.7 (2.4) ml for PURE HPMC and HPMC PLUS, and EMR specimen diameters were 10.3 (2.0) mm vs. 10.3 (1.8) mm. EGD and necropsy performed after seven days showed ulceration limited to the muscularis propria in all 38 EMR sites. Ulceration diameters were 24.4 (7.0) mm vs. 23.4 (5.0) mm. A few cases of mild periesophagitis were observed at necropsy (2/19 vs. 6/19), and histopathology revealed that mild (18/19 vs. 15/19) to moderate (1/19 vs. 3/19) inflammatory reaction surrounding the ulceration was not significantly different between the solutions. In one HPMC PLUS site with periesophagitis, a localized transmural area of necrosis was observed at histopathology, but with no endoscopic, clinical, or macroscopic evidence of perforation. The margins of resection were limited to the submucosa in all 38 specimens. Thus, in a porcine model, HPMC alone is as effective and safe for generating SFC for esophageal EMR as HPMC combined with MB and EPI.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of generating a submucosal fluid cushion in a subject, said method comprising injecting an amount of a methylcellulose solution into the submucosa of said subject, wherein said amount is effective to generate said submucosal fluid cushion.

2. The method of claim 1, wherein said methylcellulose is hydroxypropyl methylcellulose.

3. The method of claim 1, wherein said methylcellulose is carboxymethylcellulose.

4. The method of claim 1, wherein said submucosa is in the gastrointestinal tract.

5. The method of claim 1, wherein said subject is a mammal.

6. The method of claim 1, wherein said subject is a human.

7. The method of claim 1, wherein the concentration of said methylcellulose is from about 0.1% to 1.5%.

8. The method of claim 1, wherein the concentration of said methylcellulose is from about 0.3% to 1.0%.

9. The method of claim 1, wherein the concentration of said methylcellulose is 0.83%.

10. The method of claim 1, wherein said amount is from about 1 to 15 ml.

11. The method of claim 1, wherein said amount is from about 3 to 12 ml.

12. The method of claim 1, wherein said amount is from about 4 to 6 ml.

13. The method of claim 1, wherein said method further comprises performing endoscopic mucosal resection or gastrointestinal polypectomy.

14. The method of claim 1, further comprising injecting a dye or a local vasoconstrictor into the submucosa of said subject.

15. The method of claim 14, wherein said dye is methylene blue or indigo carmine.

16. The method of claim 14, wherein said local vasoconstrictor is epinephrine.

17. The method of claim 14, wherein said methylcellulose and said dye or said local vasoconstrictor are injected simultaneously into the submucosa of said subject.

18. The method of claim 1, further comprising injecting a dye and a local vasoconstrictor into the submucosa of said subject.

19. The method of claim 18, wherein said dye and said local vasoconstrictor are injected together with said methylcellulose.

20. A composition comprising a methylcellulose and a local vasoconstrictor.

21. The composition of claim 20, wherein said methylcellulose is hydroxypropyl methylcellulose.

22. A composition comprising a methylcellulose and a dye.

23. The composition of claim 22, wherein said methylcellulose is hydroxypropyl methylcellulose.

24. A composition comprising a methylcellulose, a local vasoconstrictor, and a dye.

25. The composition of claim 24, wherein said methylcellulose is hydroxypropyl methylcellulose.

26. An article of manufacture comprising:
(a) a syringe containing a methylcellulose solution; and
(b) instructions for using said methylcellulose solution to generate a submucosal fluid cushion in a subject.

27. The article of manufacture of claim 26, wherein said methylcellulose is hydroxypropyl methylcellulose.

28. The article of manufacture of claim 26, wherein said syringe further contains a dye.

29. The article of manufacture of claim 26, wherein said syringe further contains a local vasoconstrictor.

30. The article of manufacture of claim 26, wherein said syringe further contains a dye and a local vasoconstrictor.

31. The article of manufacture of claim 26, wherein said syringe contains between about 2 and about 20 ml of said methylcellulose solution.

32. The article of manufacture of claim 26, further comprising a plurality of syringes, wherein each said syringe in said plurality contains said methylcellulose solution.

33. A method of performing endoscopic mucosal resection or colonic polypectomy on a subject having a mucosal abnormality, said method comprising:
(a) generating a submucosal fluid cushion in said subject by injecting a methylcellulose solution into the submucosa of said subject; and
(b) resecting said mucosal abnormality.

34. The method of claim 33, wherein said methylcellulose is hydroxypropyl methylcellulose.

35. Use of methylcellulose in the manufacture of a medicament for creating a submucosal fluid cushion in a subject.

36. The use of claim 35, wherein said methylcellulose is hydroxypropyl methylcellulose.
